(19) United States
(12) Patent Application Publication
(10) Pub. No.: US 2001/0041853 A1
(43) Pub. Date: Nov. 15, 2001

(54) Protective cast cover

(76) Inventors: Robert W. South, Lakeland, FL (US); Olivia South, Lakeland, FL (US)

Correspondence Address: Kenneth L. Tolar, 2450 Severn Avenue, Ste. 312, Metairie, LA, 70001, US

(21) Appl. No.: 09/777,692

(22) Filed: Jan. 4, 2001

Related U.S. Application Data
(63) Continuation-in-part of application No. 08/992,826, filed on Dec. 17, 1997.

(51) Int. Cl. A61F 5/00

(52) U.S. Cl. 602/3; 602/1

(57) ABSTRACT

A cast cover configured to cover a specific portion of a user's body such as a leg, an arm, the torso or head includes an integral, elastomeric band adjacent an open end to provide a water tight seal between the cover and user's body. A leg cover has a plurality of rubber gripping members integrally protruding from a sole portion for providing traction when engaging a slippery surface. An arm cover has a plurality of gripping members on opposing surfaces of a hand portion for assisting a user in gripping objects. The gripping members allow the cover to be interchangeably used on either the left or right arm. A torso cover is also included which is uniquely configured to cover the mid torso region of a person's body. A head cover is provided which has a crown portion and a mask portion extending therefrom that is folded into the crown portion and snapped thereto to form a cap. The covers can also include intermediate elastomeric bands allowing the cover to be cut to a desired length.

This application is a continuation-in-part of application No. 08/992,826, now abandoned. The present invention relates to a water impermeable, reusable cast or bandage cover uniquely configured to protect a leg, arm, torso or head bandage.

### DESCRIPTION OF THE PRIOR ART

Typically, a cast or bandage must be kept dry, making bathing or showering difficult and cumbersome. Likewise, a user must exercise extreme caution when wearing a cast in the rain or otherwise exposing the cast to moisture. If the cast becomes wet, it may have to be replaced which is expensive and inconvenient. In the past, a cast wearer would typically cover the cast with a garbage bag or a sheet of plastic. A plastic bag or similar device is time consuming to attach and does not generally provide a water tight seal.

Numerous attempts have been made to address the above described problem. For example, U.S. Pat. No. 5,395,302 issued to Botha et al. relates to a waterproof sleeve having an open end with a sealing strip therearound for protection of a limb. The sleeve also has an elastic strap extending from the open end which is looped around a limb and fastens to itself using hook and loop fasteners.

U.S. Pat. No. 5,083,557 issued to Lennon et al. relates to a disposable boot for protecting casts or bandages. The boot has a plurality of heat seals on a sole portion to provide additional traction. The seals are formed by sealing portions of the boot sole portion to itself. The preferable material of construction is polyethylene plastic and the boot is discarded after use.

U.S. Pat. No. 5,070,630 issued to Edminson relates to a decorative cast cover fabricated with foam polystyrene to resemble a shoe or sock. The cast cover is attached to a cast using hook and loop fasteners.

U.S. Pat. No. 4,986,265 issued to Caponi discloses a protective cover for a cast comprising a polymer bag having an opening on one end thereof and an elastic edge around the periphery of the open end to secure the bag to a patient's limb. The device also has a sealing band for wrapping around the open end of the bag.

U.S. Pat. No. 4,966,135 issued to Renfrew relates to a polyurethane orthopedic cast cover designed to protect either a hand or foot. The cover is bounded by a pair of seams which minimize shearing.

U.S. Pat. No. 4,523,586 issued to Corey discloses a protective cover for a limb or cast comprising a waterproof bag with a plurality of mating fasteners on the interior surfaces adjacent an open end. The cover includes an integral flap, an edge of which is attachable to the bag exterior using hook and loop fasteners.

Although cast covers and the like currently exist in the prior art, none of these devices have the unique features and advantages of the present invention. The present invention relates to a complete set of cast covers, each specifically designed to cover a user's leg, arm, torso or head portions of the body. In addition, each cover includes means for allowing a user to quickly adjust the length of the cover to fit a particular size cast. Each of the cast covers are preferably constructed with a latex rubber or a material commonly referred to as nitrile making them water impermeable, reusable and durable. Although the patent issued to Lennon discloses a leg cover with traction on the sole portion, the device is disposable and may not be reused. Besides, the plastic sheet heat seals are tenuous and do not provide adequate traction on an extremely slippery surface. The leg cast cover according to the present invention is durable and reusable and has improved traction on the sole portion. The latex rubber cover has integral, laterally juxtaposed rubber gripping members which provide significant traction when engaging a slippery surface.

The arm cast cover has gripping members on opposing sides of the hand portion for assisting a user in gripping an object while wearing the cover on either the left or right arm. The present invention also provides uniquely configured protective covers specifically designed to cover a user's head or torso which currently do not exist in the prior art.

### SUMMARY OF THE INVENTION

The present invention relates to a set of cast covers configured to protect a user's leg, arm, torso or head. Preferably, the covers are fabricated with a latex rubber or nitrile material whereby the covers are durable, reusable and water impermeable. The leg cover comprises a hollow foot portion with a leg portion extending therefrom, the leg portion having an open end through which a user inserts a foot. The foot portion has a sole portion for engaging a planar surface such as the ground, floor, bathtub, etc. A plurality of integral rubber gripping spikes protrude from the sole portion of the leg cover to provide additional traction when the sole portion engages a wet or slippery surface. Around the periphery of the open end and integral therewith is an upper, continuous elastic band for tightly sealing the leg cover around a user's leg. Additional intermediate elastic bands are positioned along the leg portion, between the upper band and the foot portion allowing a user to cut the cover immediately above a select band to fit various size casts.

A second embodiment includes an arm cover having an open end. The arm cover comprises a hollow hand portion and a tubular arm portion extending therefrom. Integrally protruding from opposing sides of the hand portion are a plurality of rubber gripping members which assist a user in gripping and holding an object whether the cover is on the left or right arm. Around the periphery of the open end of the arm portion and integral therewith is a continuous elastic band for providing a watertight seal when the arm cover is being worn by a user. Intermediate bands are also provided allowing the arm cover to be shortened, if desired.

A third embodiment relates to a torso cover, primarily applicable to those with abdominal surgical dressings. The torso cover comprises a skirt member having open top and bottom ends. Around the periphery of the top and/or bottom end and integral therewith is a continuous elastic band for again providing a watertight seal when being worn by a user.

A fourth embodiment relates to a head cover for protecting various head and face bandages. The head cover comprises a crown portion and a mask portion extending therefrom. The mask portion has a plurality of apertures that overlay a wearer's nose, mouth and eyes. The mask portion can be folded within and attached to the crown portion to convert the device to a cap. It is therefore an object of the present invention to provide a set of cast covers configured to cover a user's leg, arm, torso or head.

It is another object of the present invention to provide a set of cast covers having gripping members thereon.

It is yet another object of the present invention to provide a set of cast covers uniquely configured to cover a variety of body portions. It is yet another object of the present invention to provide cast covers that are length adjustable to fit various size casts or bandages. Other objects, features and advantages of the present invention will become readily apparent from the following detailed description of the preferred embodiment when considered with the attached drawings and the appended claims.

### BRIEF DESCRIPTION OF THE DRAWINGS

FIG. 1 depicts the leg cover.

FIG. 2 depicts the sole portion of the leg cover with the rubber gripping members protruding therefrom.

FIG. 3 depicts the arm cover.

FIG. 4 depicts the torso cover.

FIG. 5 depicts the head cover.

FIG. 6 depicts the head cover with the mask portion folded into the crown portion to form a cap.

FIG. 7 depicts a length adjustable leg cover.

FIG. 8 depicts a length adjustable arm cover.

### DESCRIPTION OF THE PREFERRED EMBODIMENT

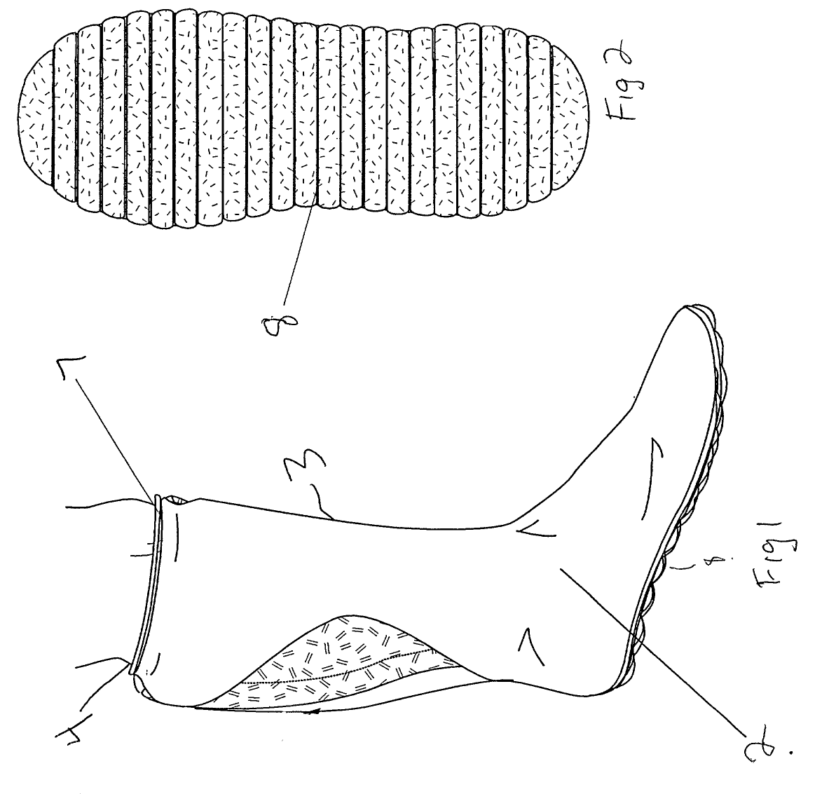

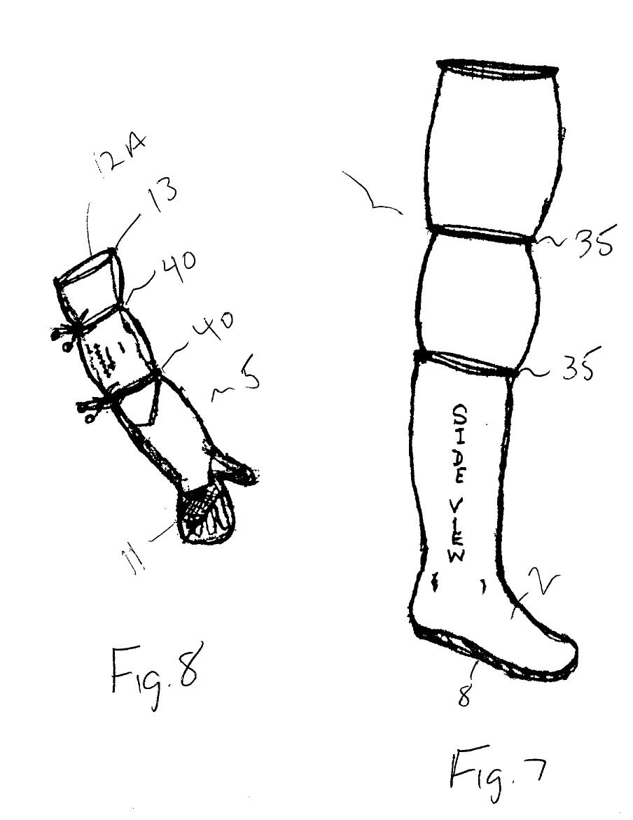

Referring now to FIGS. 1 through 6, the present invention relates to protective cast covers for covering various portions of a user's body. The arm and leg covers are substantially similar with each having a limb receiving portion and a terminal portion for receiving the end of a user's limb such as a hand or foot. FIGS. 1 and 2 depict the first embodiment of the present invention, specifically the leg cover. The leg cover includes a hollow foot portion 2 with a leg portion 3 extending therefrom. The leg portion 3 has an open end 4 through which a user may insert his or her foot. Around the periphery of the open end and integral therewith is a continuous elastic upper band 7 for tightly securing the open end around a user's leg. As depicted in FIG. 7, intermediate bands 35 can be positioned between the upper band and the foot portion whereby a user can cut the leg portion immediately above a select intermediate band to shorten the cover to fit about a particular size cast. When the cover is cut in such fashion, the select intermediate band replaces the upper sealing band. The foot portion includes a lower, sole portion. Integrally protruding from the sole portion are a plurality of laterally juxtaposed rubber gripping members 8 for providing additional traction when the sole portion is engaging a wet surface.

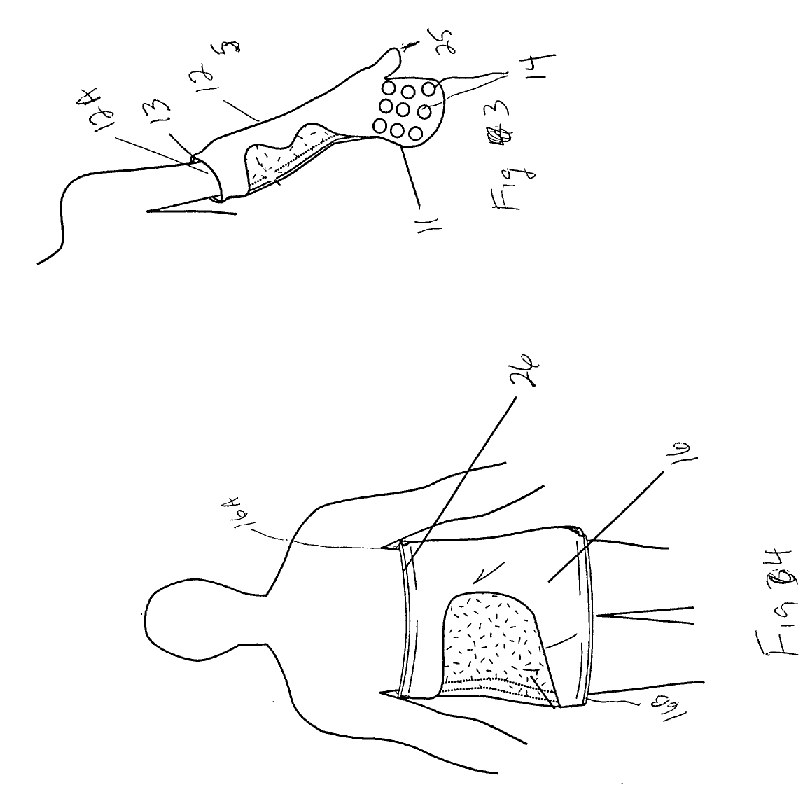

FIG. 3 depicts a second embodiment of the present invention relating to an arm cover 5. The arm cover 5 includes a hand portion 11 having upper and lower surfaces for covering a user's hand with a sleeve portion 12 extending therefrom. The sleeve portion 12 has an open end 12A with an integral continuous elastic band 13 around the periphery thereof. On both the upper and lower surfaces of the hand portion 11 are a plurality of rubber gripping members 14 for assisting a user in grasping objects while taking a shower. The gripping members 14 are provided on both the upper and lower surfaces of the hand portion so that the arm cover may be interchangeably used on either the left or right arm while gripping members will overlay the user's palm. Preferably, the hand portion 11 has a thumb section 25 for receiving a user's thumb. As depicted in FIG. 8, the arm cover can also include intermediate bands 40 allowing the size of the cover to be adjusted as described above.

FIG. 4 depicts a third embodiment of the present invention and relates to a torso cover 16. The torso cover 16 comprises a skirt member, having open top 16A and bottom 16B ends, and which is configured to tightly surround a user's torso below the armpits. Around the periphery of the open top end 16A (and possibly the bottom end) and integral therewith is a continuous elastic band 26 for tightly sealing the top end around a user's torso. The torso cover is particularly applicable for those who have undergone recent heart surgery, back surgery or similar procedures which result in incisions on the mid-torso area. The torso cover may also include intermediate bands allowing the size of the cover to be modified.

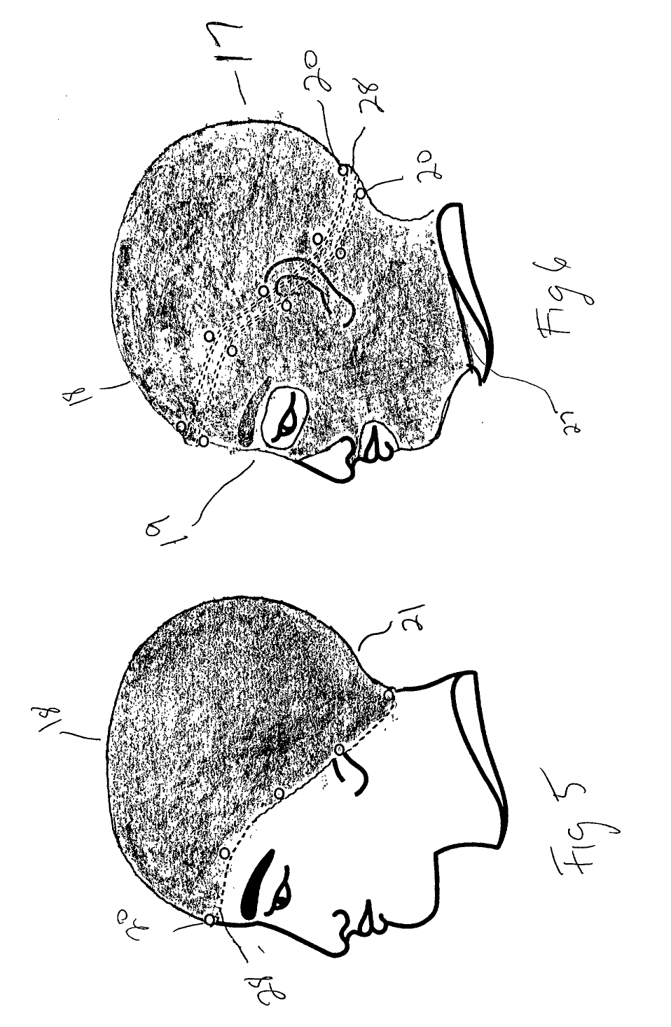

FIGS. 5 through 6 depict a fourth embodiment of the present invention. A head cover 17 is shown and comprises a crown portion 18 with a mask portion 19 extending therefrom. The mask portion has an open end 27 for receiving a user's head. Between the mask portion 19 and crown portion 18 is a fold line 28 with mating snaps 20 on opposing sides thereof. The snaps 20 allow the mask portion 19 to be folded into the crown portion 18 and snapped thereto thereby converting the head cover to a cap member 21 as depicted in FIG. 5. The cap member 21 may be used to cover head bandages, incisions and the like on the top portion of the head. The mask portion 19 has a plurality of openings that overlay a user's eyes, nose and mouth. The mask portion 19 may be used to cover face bandages and similar dressings.

As discussed above, each of the above described covers are preferably constructed with a latex rubber or nitrile material providing a water impermeable yet reusable material. However, as will be readily apparent to those skilled in the art, many other suitable materials will suffice. The elastic bands on the arm, leg and torso covers are constructed and dimensioned to tightly engage a user's skin to provide a water tight seal. The torso cover may also have an integral elastic band at the bottom end to provide additional protection against moisture leaking therein.

Although there has been shown and described the preferred embodiment of the present invention, it will be readily apparent to those skilled in the art that modifications may be made thereto which do not exceed the scope of the appended claims. Therefore, the scope of the invention is only to be limited by the following appended claims.

What is claimed is:

1. A cast cover comprising:
a limb receiving portion having an open end, said limb receiving portion dimensioned to tightly surround a limb cast;
a terminal portion for receiving an end of a user's limb, said terminal portion having an upper surface and a lower surface;
a plurality of gripping members integrally protruding from said lower surface of said terminal portion for frictionally engaging a wet surface;
a continuous integral elastic upper band peripherally disposed about said open end for providing a water tight seal between the open end and a user's limb.

2. A cast cover according to claim 1 wherein said terminal portion is a foot portion for receiving a user's foot, the lower surface of which defines a sole portion wherein said gripping members are lateral, juxtaposed and integrally protrude from said sole portion.

3. A cast cover according to claim 2 wherein said cast cover is fabricated with a nitrile material thereby providing a durable, reusable water impermeable cast cover.

4. A cast cover according to claim 1 wherein said terminal portion is a hand portion further having a plurality of gripping members integrally protruding from said upper surface for assisting a user in grasping slippery objects whereby said cover is interchangeably wearable on a user's right and left arm.

5. A cast cover according to claim 1 wherein said limb portion includes a plurality of intermediate elastic bands positioned between said upper band and said terminal portion whereby a user cuts the cover immediately above a select band to adjust the length of said cover.

6. A cast cover for protecting incision coverings on a user's torso comprising:
a cylindrical skirt component, said skirt component dimensioned to tightly surround a user's torso, said skirt component having two opposing open ends;
a continuous elastic band integral with the periphery of one of said open ends for providing a water tight seal between said skirt and the torso.

7. A cast cover according to claim 6 wherein said torso cover is fabricated with a nitrile material.

8. A wound cover for selectively covering a user's head:
a hollow crown portion for covering a top portion of a user's head;
a hollow mask portion extending from said crown portion with a fold line therebetween, said mask portion having a plurality of openings thereon for overlaying a wearer's eyes, nose and mouth, said mask portion further having an opening through which a wearer's head is inserted;
a plurality of mating snaps on opposing sides of said fold line whereby said mask portion is folded about said fold line and secured within said crown portion to selectively form a cap.

9. A cast cover according to claim 8 wherein said head cover is fabricated with a nitrile material.
